(19) United States
(12) Patent Application Publication
(10) Pub. No.: US 2003/0152990 A1
(43) Pub. Date: Aug. 14, 2003

(54) Compositions and methods for detecting redox-active molecules in solution

(76) Inventors: Stephen E. Creager, Central, SC (US); Marla French, Derwood, MD (US); Philip T. Radford, Roebuck, SC (US)

Correspondence Address: DORITY & MANNING, P.A., POST OFFICE BOX 1449, GREENVILLE, SC, 29602-1449, US

(21) Appl. No.: 10/368,820

(22) Filed: Feb. 19, 2003

Related U.S. Application Data
(63) Continuation of application No. 09/805,549, filed on Mar. 13, 2001, now abandoned.
(60) Provisional application No. 60/192,211, filed on Mar. 27, 2000.

(51) Int. Cl. C12Q 1/68; G01N 33/53

(52) U.S. Cl. 435/6; 435/7.1; 205/777.5

(57) ABSTRACT

An electrochemical amplification scheme for detecting very small amounts of redox-active molecules is disclosed. The reaction involves “recycling” of oxidized analyte molecules by way of a solution-phase electron exchange reaction with a sacrificial electron donor. The scheme relies heavily upon the action of a selective monolayer coating on the electrode that suppresses direct oxidation of the sacrificial donor but facilitates the oxidation of analyte molecules. The method is particularly useful for detection of hydroxymethylferrocene at a dodecanethiolate-coated gold electrode with ferrocyanide as the sacrificial electron donor.

### REFERENCE TO PREVIOUS APPLICATION

This application claims priority from previously filed Provisional Application No. 60/192,211 filed on Mar. 27, 2000.

### FIELD OF THE INVENTION

The invention is directed to a new method and system for detecting redox-active molecules in solution at low concentration levels.

### BACKGROUND OF THE INVENTION

Amperometric detection of redox active molecules in solution is used to detect very small amounts of a substance or chemical in a solution via oxidation or reduction of that chemical, usually at an electrode. This type of analysis is useful in forensic chemistry, clinical chemistry, and many other applications in which a trace amount of material is to be discerned in a solution. However, such detection strategies are inherently limited by the fact that each analyte molecule (i.e. the trace molecule being detected) can accept or donate only a relatively small number of electrons on oxidation or reduction. Because of this limitation, and because of the noise inherent in measuring very small currents (i.e. currents of less than a few hundred femtoamperes are difficult to measure reliably), detection limits for amperometric detection are often not nearly as low as would be desired.

Most strategies for improving the detection limits in such processes involve some form of analyte recycling, either physically (i.e. regeneration at a second electrode) or chemically (regeneration via a coupled chemical reaction). Recycling strategies in general are able to improve detection limits because they amplify the signal, thereby increasing sensitivity without increasing the background noise.

A prior art detection scheme that employs two electrodes placed closely together has been used, in which the analyte is regenerated by way of a second electrode that is placed in very close proximity to the detection electrode. In this method, amplification depends upon diffusion time between electrodes, and the molecules must travel in a “circuit” at in order to carry out the detection method. It is very difficult to achieve high amplification in these systems, because the relatively short residence time in the detector cell does not allow for many recycling events. Amplification factors greater than 10× are rare using such two electrode methods.

Another prior art detection scheme employs a chemical reaction to regenerate the analyte. One characteristic feature of most chemical recycling strategies is that they require the presence of a reagant which can react with the oxidized or reduced form of analyte chemically, but for which direct reaction at the electrode is suppressed. The recycling scheme typically depends upon the direct reaction of the reagant at the electrode being inhibited, usually because it is kinetically too slow to occur as a direct electrode reaction. It has often been necessary to use some sort of catalyst (i.e. redox enzyme) to facilitate the recycling. Unfortunately, reactions that depend upon catalysts are very specific, and proceed too slowly, which can limit the amplification factors that can be achieved using such methods.

What is needed in the industry is a relatively simple redox amplification method which can provide a large signal enhancement for amperometric electrochemical detection of redox molecules in both quiescent solutions and flowing streams. A method that uses relatively simple molecules and requires no enzyme catalyst would be very desirable.

### SUMMARY OF THE INVENTION

A new signal amplification scheme is employed in one aspect of the invention for ultrasensitive amperometric electrochemical detection of redox-active molecules in quiescent solution. The invention may be employed in many different environments, including in flowing streams. The method, in one embodiment, is based upon a continuous regeneration of electrochemically oxidized analytes by reaction with a sacrificial electron donor in solution. The method, in one embodiment, utilizes a selective coating on the electrode. The electrode may exhibit properties that facilitate relatively facile electrooxidation of analyte. However, the invention also serves to inhibit electro-oxidation of the sacrificial electron donor on the electrode.

The method employs a solution having an analyte therein, and an electrode in electrical communication with the solution. A sacrificial reagant is also employed in the solution. A charge is transferred from the sacrificial reagant to the analyte. Furthermore, a charge is s transferred from the analyte to the electrode, thereby generating an electrical signal. The charge transfer is repeated several times, facilitating amplification of the signal, and the amplified electrical signal then may be measured to determine the approximate concentration of the analyte in solution.

In one aspect of the invention, a method is employed wherein the electrode further comprises a coating layer adjacent to the electrode, the coating layer having an upper surface in contact with the solution and a lower surface in electrical communication with the electrode, the coating layer being capable of conducting a charge from the analyte to the upper surface to the electrode.

One embodiment of the invention employs an analyte which is a derivative of ferrocene. One analyte that works well in the method of the invention is hydroxymethylferrocene (HMFc). The method may be employed with a sacrificial reagant comprising a ferrocyanide. The method may amplify the signal by a factor of at least one thousand, and in some embodiments, a detection of as little as 60,000 or less injected molecules may be detected.

One embodiment of the invention employs a method of detecting relatively low levels of an analyte in solution by amplifying amperometric signals for a solution having an analyte therein, the analyte comprising a ferrocene derivative. A sacrificial reagant is employed in the solution, and a charge is transferred from the sacrificial reagant to the analyte, and from the analyte to the upper surface of the coating layer. Then, a charge is transferred from the coating layer to the electrode, thereby generating an electrical signal. By repeating this transfer numerous times, an amplified signal is generated.

The method may be employed in which the ferrocene derivative further comprises a tagged biomolecule, the biomolecule being selected from the group of comprising: proteins, enzymes, oligopeptides, oligonucleotides, and antibodies. In some applications, the ferrocene derivative comprises a ferrocene moiety which is attached to a strand of deoxyribonucleic acid (DNA).

### BRIEF DESCRIPTION OF THE DRAWINGS

A full and enabling disclosure of this invention, including the best mode shown to one of ordinary skill in the art, is set forth in this specification. The following Figures illustrate the invention:

FIG. 1 is a schematic showing a prior art two electrode method of detection in which a second electrode is placed in very close proximity;

FIG. 2 shows a typical prior art method employing sacrificial reagants and analyte;

FIG. 3 depicts a prior art system in which an analyte does not participate directly in the charge-transfer reaction, but rather serves as a catalyst to generate a quantity of a species that can participate in a charge-transfer reaction;

FIG. 4 shows the electrochemical amplification method and used with one embodiment of the invention in which a coating or layer on top of an electrode receives an electrical charge from an analyte;

FIG. 5 depicts a further system of the invention in which a ferrocyanide is used as a sacrificial reagant, a ferrocene is employed as an analyte, and the coating on the electrode is an alkanethiol monolayer on gold;

FIG. 6 is a schematic showing one method in which the invention may be employed wherein a separation column carries the solution from a fluid delivery system to a detector cell;

FIG. 7 is a schematic showing an FIA apparatus that employs an HPLC (high pressure liquid chromatography) system capable of using a reference electrode and a working electrode, in one embodiment of the invention;

FIG. 8 shows an example of a ferrocene tagged biomolecule and system that can be used in the practice of the invention;

FIG. 9 shows one embodiment of the invention that employs a DNA strand that is attached to a ferrocene cluster; and

FIG. 10 is a schematic of one embodiment of the invention that uses a target DNA strand.

### DETAILED DESCRIPTION OF THE INVENTION

Reference now will be made to the embodiments of the invention, one or more examples of which are set forth below. Each example is provided by way of explanation of the invention, not as a limitation of the invention. In fact, it will be apparent to those skilled in the art that various modifications and variations can be made in this invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention, which broader aspects are embodied in the exemplary constructions.

A new signal amplification scheme for ultrasensitive amperometric electrochemical detection of redox-active molecules in quiescent solution and in flowing streams is described. The method is based upon a continuous regeneration of electrochemically oxidized analytes by reaction with a sacrificial electron donor in solution. The method utilizes a selective coating on the electrode that is chosen to have properties which allow for relatively facile electrooxidation of analyte, but which also inhibits electrooxidation of the sacrificial electron donor. Ultrasensitive detection of hydroxymethylferrocene (HMFc) as a model analyte using ferrocyanide as the sacrificial electron donor may occur at a dodecanethiol-coated gold electrode, as one example.

Signal amplification factors of several hundred to several thousand are obtained in flow-injection mode for analyte injections in a concentration range between 10⁻⁴ and 10⁻⁷ M where peaks can be discerned both with and without amplification. Even higher amplification factors are estimated to be reached for analyte concentrations below approximately 10⁻⁸ M, for which peaks without amplification are typically undetectable. Amperometric detection of 60 million injected HMFc analyte molecules (corresponding to either a 1 [symbol] L injection at 10⁻¹¹ M or a 1.0 mL injection at 10⁻¹³ M) is also possible using the method in flow-injection mode, as further described below.

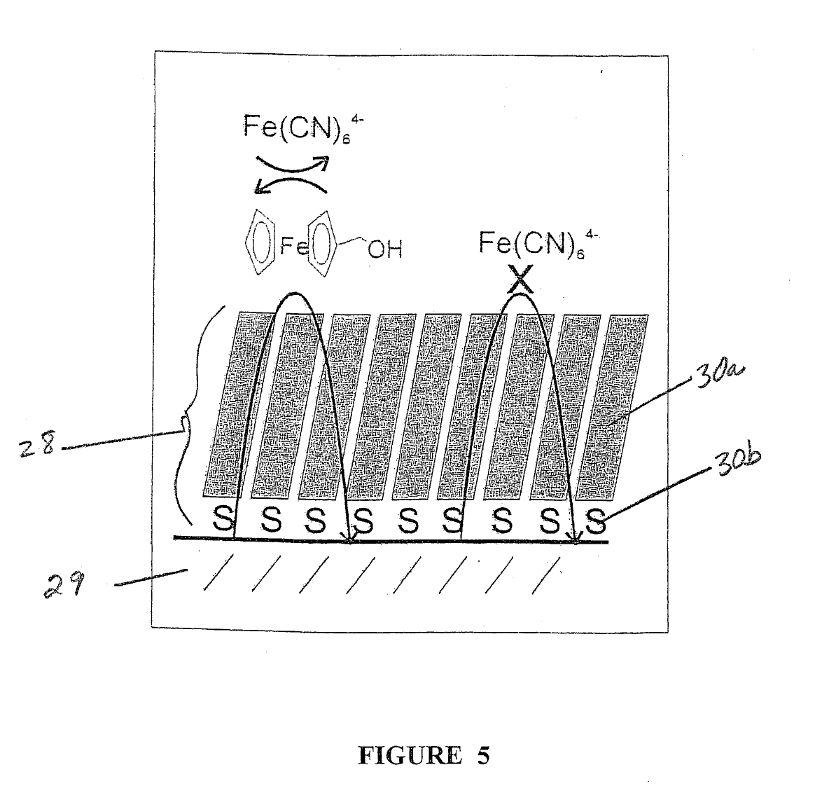

In this invention, a relatively simple redox amplification scheme is provided that can provide large signal enhancements for amperometric electrochemical detection of redox molecules in both quiescent solutions and flowing streams. The method employs redox molecules and requires no enzyme catalyst. As is usually the case in such schemes, it does require that the medium include a quantity of a sacrificial electron donor that will serve to recycle the oxidized redox molecules via a solution-phase electron-exchange reaction. A sample amplification scheme with hydroxymethylferrocene as analyte and ferrocyanide as the sacrificial electron donor is illustrated in FIG. 5. The electrode reactions in this scheme are as follows:

HMFc→HMFc⁻

Fe(CN)₆⁴⁻+HMFc→Fe(CN)₆³⁻+HMFc

The reaction between hydroxymethylferricenium and ferrocyanide is approximately thermoneutral (the formal potentials are less than 10 mV apart) but typically are relatively rapid since both molecules are characterized by relatively rapid electron self-exchange rate constants. Thus, this pair of reactants is well suited to an electrochemical amplification scheme for detecting ferrocene derivatives and ferrocene-tagged analytes.

In the application of the invention, it is possible to enhance amperometric signals for redox active molecules without enhancing noise levels. This enables very low limits of detection. The method is also selective for specific categories of redox active analytes. The invention may be employed in flowing streams, or in other environments. Furthermore, the invention can be employed to use tagged analytes, as further described below.

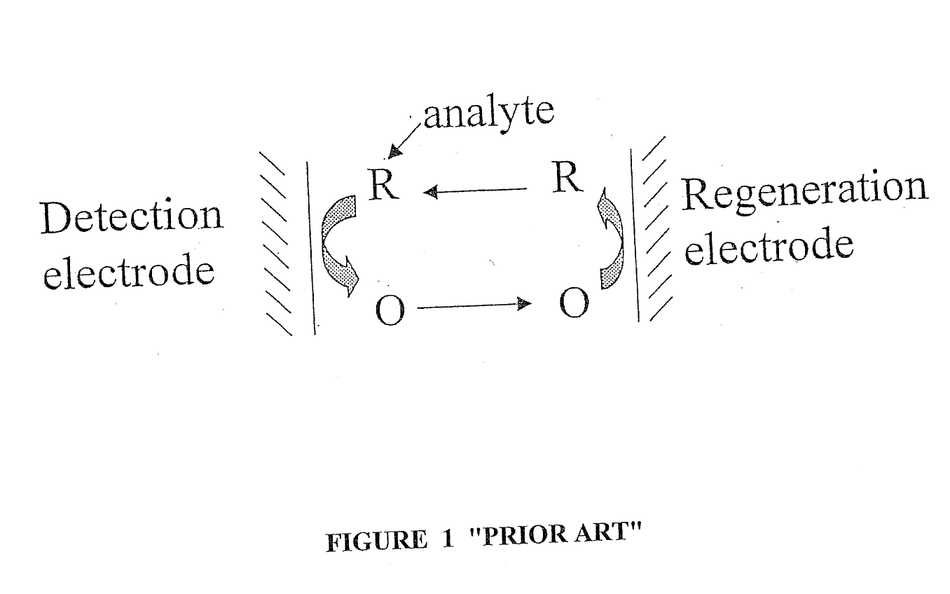

For comparison purposes, FIG. 1 shows a typical prior art detection scheme that employs two electrodes placed closely together. In this method, amplification depends upon diffusion time between electrodes. It is very difficult to achieve high amplification in these systems, because the relatively short residence time in the detector cell does not allow sufficient time for many recycling events to occur. Amplification factors greater than 10× are rare using such two electrode methods.

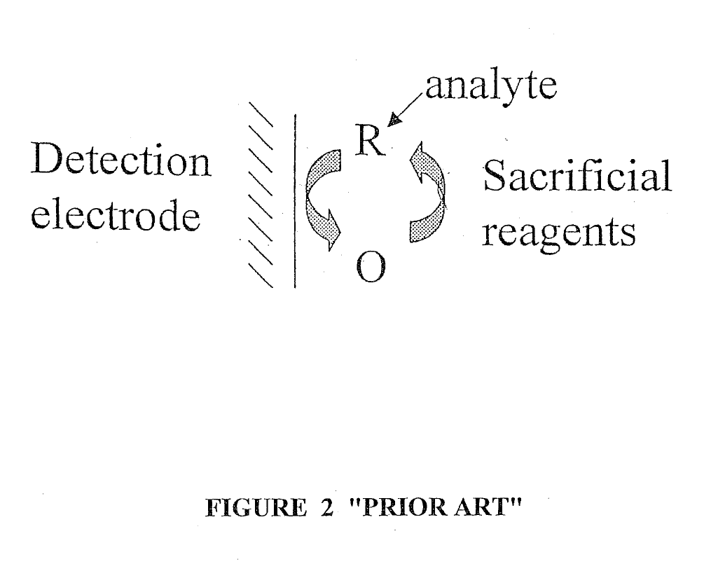

In FIG. 2, a prior art method is shown that provides for regeneration of the analyte by way of a chemical reaction with a sacrificial reagant. The process usually requires that the sacrificial reagant not itself be reactive with the electrode. Often, such methods which have been used in the prior art require enzyme catalysis, which can be slow and which often shows poor reproducibility and poor long term stability.

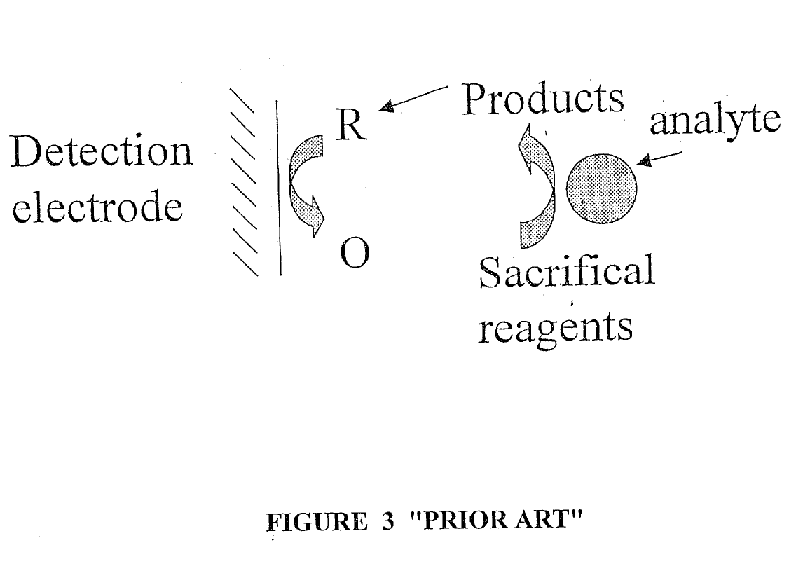

FIG. 3 shows another prior art scheme that uses an analyte that converts an oxidized reagant to a reduced reagant, so that the reagant can work to carry out a reaction at an electrode surface. Unfortunately, this sort of scheme requires a reaction that takes place very close to the electrode surface, otherwise, the products are transported away from the electrode. Most of such prior art schemes also require enzyme catalysts which are undesirable for reasons provided above.

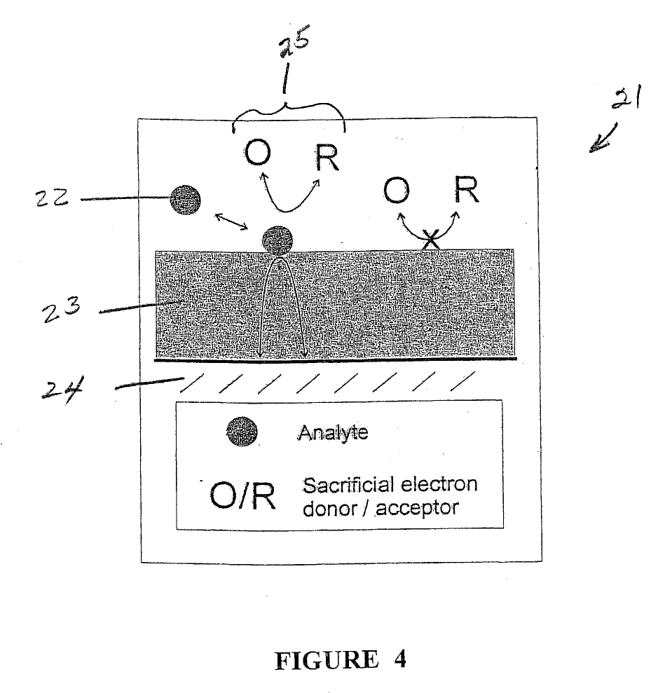

In FIG. 4, a reaction methodology of this invention is shown that employs a surface selective reaction 21. Surprisingly, it has been discovered that by employing the analyte on or near the surface of a coating layer 23 fixed to an electrode 24, it is possible to facilitate the oxidation of the sacrificial reagant 25 on the analyte, but inhibit the oxidation of the reagant on the coating layer 23. That is, the sacrificial reagant 25 will react upon the analyte, but not upon the coating layer 23 of electrode 24, thereby providing the selectivity to amplify the signals generated by transmission of the charge from the analyte 22 to the electrode 24.

FIG. 5 shows one application of the invention that uses a ferrocyanide as the highly charged sacrificial reagant and a ferrocene as the analyte. A monolayer 28 is shown attached to the electrode 29, and the reaction or charge transfer from the ferrocyanide to the ferrocene, followed by charge transfer from the ferrocene to the electrode 29. An “oily” monolayer 28 is comprised of a series of hydrocarbon alkane chains 30a, attached to sulfur groups 30b. The sulfur groups are directly adjacent to the electrode 29.

The method illustrated in FIG. 5 requires that direct ferrocyanide oxidation at the electrode be inhibited (i.e. the “X” indicates that it is inhibited or minimized) but that direct hydroxymethylferrocene oxidation at the electrode occurs relatively rapidly. Coating the electrode with a self-assembled alkanethiolate monolayer 28, as one option, may provide this surprising reactivity pattern. Monolayers such as monolayer 28 can serve as excellent barrier layers for preventing oxidation of highly charged and well-solvated metal complexes such as ferrocyanide, but they are usually not good barriers for preventing oxidation of neutral or poorly-solvated molecules such as most ferrocene derivatives.

In a preferred embodiment, the selectivity of a dodecanethiolate monolayer on gold is may be used to suppress the direct oxidation of ferrocyanide, thereby enabling the signal amplification scheme for hydroxymethylferrocene detection. Thus, one preferred monolayer is dodecanethiolate, and one electrode that is preferred is a gold electrode.

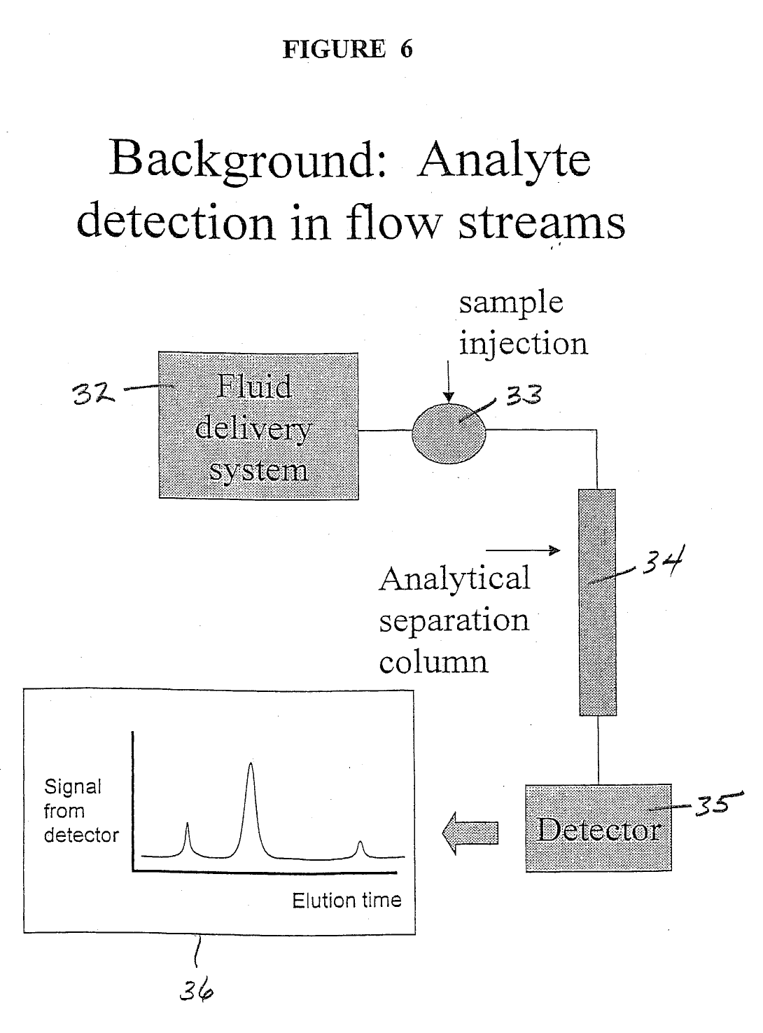

In FIG. 6, one application of the invention is shown comprising a fluid delivery system 32. A sample comprising analyte is injected at sample injection point 33, and flows along an analytical separation column 34 to a detector 35. The detector 35 is operably connected to an output means, such as for example a data output 36.

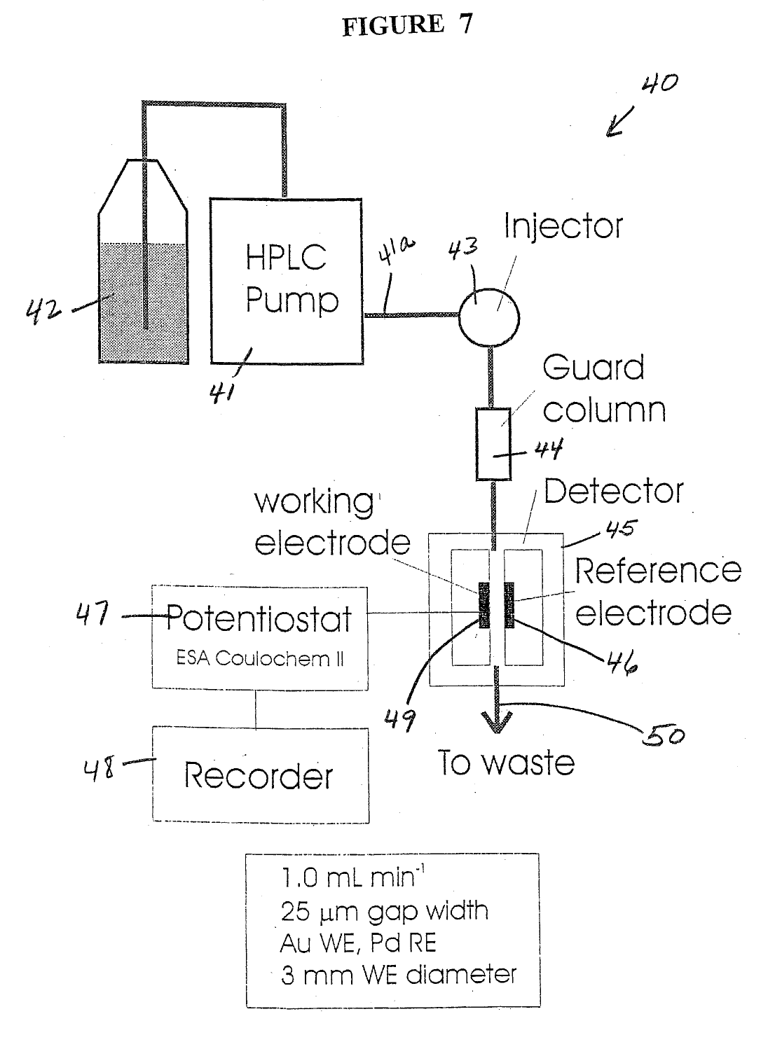

FIG. 7 shows a method of applying the compositions in the practice of the invention in which an FIA apparatus (“FIA” denotes flow injection analysis) is configured to supply a solution 42 by way of a HPLC pump 41 (or other fluid delivery system) along line 41a. Injector 43 is the entry point for the analyte, and a guard column 44 is used. A detector 45 is comprised of a reference electrode 46 (which may be comprised of palladium, Pd) and a working electrode 49 comprised of a metal such as gold. Further, a flow exit 50 goes to waste. A potentiostat 47 is operably connected to an output means such as recorder 48. In the practice of the invention, a flow of about 1.0 ml/min with a 25 micrometer gap width is possible. The working electrode 49 preferably has a diameter of about 3 mm.

A new electrochemical amplification scheme for detecting very small amounts of redox-active molecules is possible in the application of the invention. The reaction involves “recycling” of oxidized analyte molecules via a solution-phase electron exchange reaction with a sacrificial electron donor. The method relies upon the action of a selective monolayer coating on the electrode which suppresses the direct oxidation of the sacrificial donor but permits the facile oxidation of analyte molecules.

The scheme is demonstrated below for detection of hydroxymethylferrocene at a dodecanethiolate-coated gold electrode with ferrocyanide as the analyte, but other compounds could be used in the application of the invention, and the invention is not limited to the specific examples shown herein.

### EXAMPLE 1

In one application of the invention, an analyte may be measured by amplifying the electrical signal that is passed to the electrode. A buffer (pH 5) used in both quiescent solution and flowing stream experiments is composed of 0.10 M sodium perchlorate (Alfa Aesar), 0.10 M acetic acid (Fisher) and 0.18 M sodium acetate trihydrate (Alfa Aesar). Hydroxymethylferrocene (HMFc) was purchased from Strem, a chemical supplier known to persons of skill in the art. Sodium ferrocyanide decahydrate (Na₄Fe(CN)₆:10H₂O) was obtained from Fluka Chemika Company and mixed with the pH 5 buffer to make a 10⁻⁴ M solution of ferrocyanide in the buffer. Due to the sensitivity of ferrocyanide in solution to reaction with dissolved oxygen, this buffer solution was prepared fresh daily. Mineral acids used to mix the dilute aqua regia (1:3:4 HNO₃(conc.):HCl(conc.):H₂O by volume) for etching electrodes were obtained from Fisher Scientific. A 1-Dodecanethiol for monolayer preparation was purchased from Aldrich Chemical. Reagents were used as received from their respective manufacturers. All water for aqueous solutions was deionized using a Barnstead Nanopure system to a resistivity of about 17 megohm cm.

A 10 mM HMFc stock solution in ethanol was prepared by adding a measured amount of solid HMFc to ethanol in a 10 mL volumetric flask. 10 [symbol] L of the 10 mM HMFc stock solution was diluted with 90 [symbol] L of buffer using two Wheaton Socorex Micropipetes (10 [symbol] L and 100 [symbol] L) to make a 10⁻³ M HMFc solution. The solution was placed in a new 1.5 mL polypropylene microcentrifuge tube (Fisher) and inverted and shaken 30 times to facilitate mixing. The second dilution was performed by taking 10 [symbol] L of the 10⁻³ M HMFc solution and adding 90 [symbol] L of the buffer to make a 10⁻⁴ M HMFc solution. This process was repeated until all the standards, 10⁻³ M to 10⁻¹³ M HMFc, were prepared. Two sets of HMFc standards were made, one using pure buffer (no ferrocyanide) and another using buffer containing 10⁻⁴ M ferrocyanide. Ferrocyanide was included in both the running buffer and the injected solution in FIA experiments to avoid small changes in the ferrocyanide concentration as the injected plug passes through the detector.

Electrodes for cyclic voltammetry were constructed of 0.127 mm Au wire (Alfa, Premion grade>99.999% pure), encased in epoxy (Epon 825, Shell Chemical Company) that was crosslinked with 1,4-diaminocyclohexane (Aldrich Chemical Company) and hardened for 3 hours at about 80 degree C. The electrodes were then sanded and successively polished by hand using 25, 5, and 1 micron alumina with rinsing and a 1 minute etch in dilute aqua regia between each polishing step. The 3 mm diameter gold electrode from the flowcell was mounted in a Minimet 1000 Grinder/Polisher and polished using the same process. This treatment produced an uncontaminated and stable gold foundation upon which the monolayers could be formed. After rinsing with water and isopropanol, the electrodes were suspended in a 1.0 mM solution of alkanethiol in ethanol for 20-24 hours to form the monolayer.

Cyclic voltammetry was performed using a computer-based CH-Instruments model 660 electrochemical workstation. A three-electrode configuration with a platinum wire auxiliary electrode and a Ag/AgCl/sat. A KCl reference electrode in pH 5 buffer electrolyte was used. The potential was swept over a range from +0.0 V to +0.7 V at a scan rate of 0.1 V sec⁻¹.

The flow injection apparatus consisted of an ISCO model 2350 HPLC pump fitted with a Rheodyne model 7125 injection valve connected to an ESA CouloChem II electrochemical detector with an ESA model 5041 flow cell. The pump delivered buffer at a preset flow rate of about 1 mL min⁻¹. The buffer was continuously degassed by bubbling with house nitrogen prior to pumping through the FIA system.

Hydrodynamic voltammetry was performed in the flow cell by setting the applied potential to the desired value, allowing the current to stabilize and injecting a series of 1 [symbol] L aliquots of a [symbol] ×10⁻⁶ M HMFc solution both with and without [symbol] ×10⁻⁴ M ferrocyanide present. The voltammetry was conducted at both a bare gold electrode (etched for 1 minute in dilute aqua) and at a dodecanethiol-coated gold electrode. The etching step serves to clean the gold electrode prior to coating with the dodecanethiol monolayer. It is a useful step, but not a required component of this particular embodiment of the invention. The potential was stepped in +0.1V increments for subsequent injections. Current-time traces following injections were recorded using a Lineseis Ly16100-11 chart recorder. Following each injection at progressively positive potentials, a repeat injection at +0.1 V vs. reference (an internal palladium-hydrogen electrode) was made to establish whether the changes in response with increasingly positive applied potential were reversible.

Current vs. time traces for different HMFc concentrations were recorded for the series of injections of progressively more dilute HMFc solutions in pure buffer (ferrocyanide free), and also in buffer solutions containing 1×10⁻⁴ M ferrocyanide. A short guard column (SiO₂-packed) was included between the injection port and the detector to help damp the pressure pulse originating from the injection event. Detection was performed at an applied potential of +0.4 V vs. reference for injections containing between 10⁻⁴ M and 10⁻¹³ M HMFc for both buffer series.

The ferrocyanide electrooxidation reaction was effectively “blocked” by the monolayer. This effect reflects the fact that the monolayer prevents close approach of ferrocyanide ions to the electrode, which in turn causes the standard electron-transfer rate constant for ferrocyanide to be greatly diminished relative to that at an uncoated electrode. The slight rise in current at potentials more positive than approximately +0.6 V probably reflects the onset of long-range electron tunneling as a mechanism for oxidizing ferrocyanide through the monolayer. This is an inherently slow process, as indicated by the fact that such a large positive overpotential is required to drive the oxidation. The oxidized HMFc molecules have been recycled by the solution-phase electron-transfer reaction between oxidized HMFc and ferrocyanide.

### EXAMPLE 2

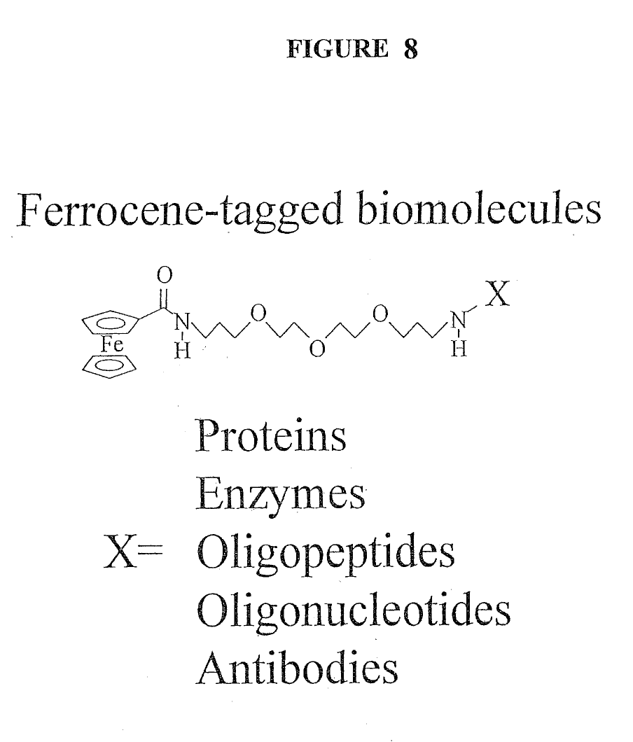

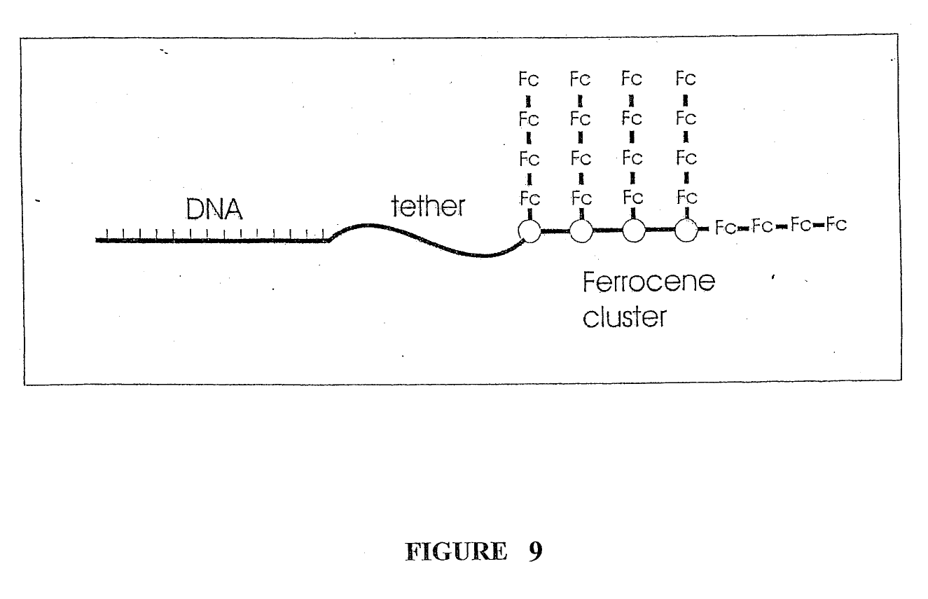

In this example, ferrocene tagged biomolecules may be detected at unusually low concentration levels. As seen in FIG. 8, for example, biomolecules may be tagged by attachment to a ferrocene derivative. Proteins, enzymes, oligopeptides, oligonucleotides, antibodies and other compounds may be detected. Tagging could be accomplished by linking a ferrocene group or other redox molecule suitable for catalytic amplified detection to the biomolecule via a tethering chain, for example the oligo-ethylene glycol chain illustrated in FIG. 8. Alternatively, a cluster of ferrocenes or other catalytic redox molecules could be linked via a tethering chain to a biomolecule, as illustrated in FIG. 9 for a cluster of ferrocenes linked to DNA. Tagging with ferrocene groups would probably be accomplished prior to the biomolecule being injected into a flow stream or separation column. With other tagging molecules it is possible to accomplish tagging after the molecules elute from a separation column. In this implementation of the invention, selective detection of tagged molecules would be accomplished by virtue of the fact that only the biomolecule(s) of interest have been tagged.

### EXAMPLE 3

In this example, amplified electrochemical detection of ferrocene-tagged bioaffinity agents could be used to detect the binding of an (unlabeled) complementary molecule to the bioaffinity agent via a change in the elution characteristics of the labeled bioaffinity agent after binding of the complement. For example, if the labeled bioaffinity agent is an antibody, then the elution time on a chromatographic separation, or the electrophoretic mobility in an electrophoretic separation, would be different depending upon whether the labeled antibody was bound to it's complementary antigen. Similarly, a labeled oligonucleotide would exhibit different elution characteristics depending upon whether or not it was bound to it's complement by sequence-specific base-pairing hybridization. A change in elution characteristics on exposure to a sample being tested would be indicative of the presence of unlabeled complement in the sample.

### EXAMPLE 4

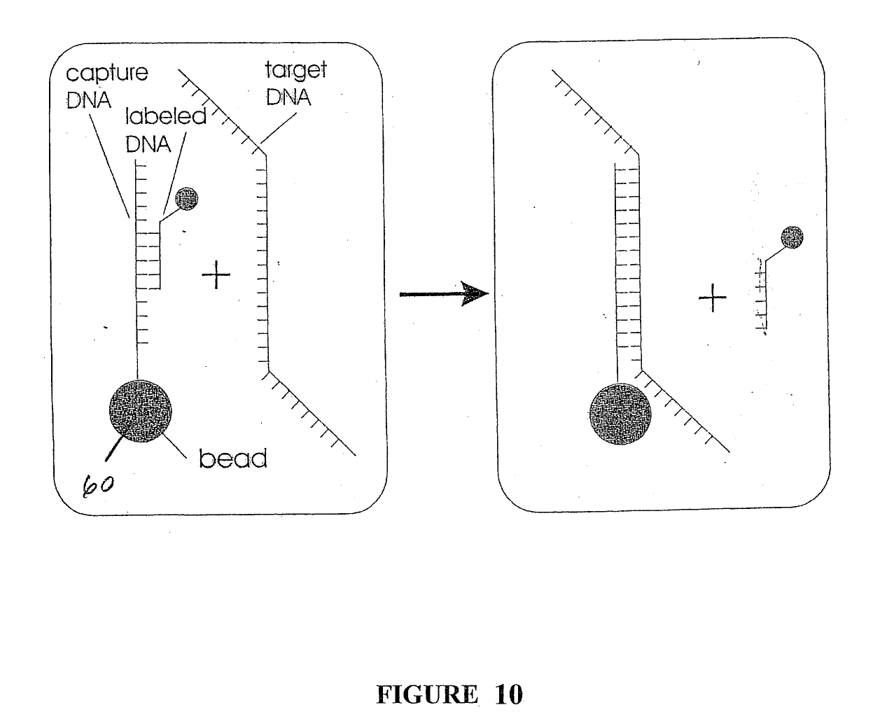

In this example, amplified electrochemical detection of ferrocene-labeled biomolecules is used to detect the binding of target analyte molecules onto a surface via displacement of the labeled molecules from a surface. The surface is pre-loaded with weakly-bound labeled biomolecules, which are subsequently displaced from their binding sited by the unlabeled target molecules when they bind to the surface. The released labeled molecules are ultimately detected in solution via amplified electrochemical detection. The idea is illustrated in FIG. 10 for the specific case of displacement of a ferrocene-labeled oligonucleotide that is initially held via sequence-specific base-pair hybridization to a complementary capture DNA strand that is immobilized onto a surface, for example a solid bead. Upon exposure to a long strand of target DNA that is complementary to the capture DNA strand, the labeled DNA is released and subsequently detected via amplified electrochemical detection, perhaps via injection into a flow stream as described above.

### EXAMPLE 5

In this example, amplified electrochemical detection is used to detect simple redox-active molecules suitable for amplification (i.e., a ferrocene that is not attached to a biomolecule) that are released from an enclosed space (for example the inside of a phospholipid vesicle). The objects that include the trapped redox molecules (hereafter referred to as the vesicles) are captured at a surface by a biospecific interaction, for example base-pair-specific nucleic acid hybridization to nucleic acid tags on the vesicle. Upon release of the redox molecules from the vesicle, the redox molecules are detected with high sensitivity using the electrochemical amplification method.

It is understood by one of ordinary skill in the art that the present discussion is a description of exemplary embodiments only, and is not intended as limiting the broader aspects of the present invention, which broader aspects are embodied in the exemplary constructions. The invention is shown by example in the appended claims.

1. A method of detecting an analyte in solution, comprising:
(a) providing a solution having an analyte therein;
(b) providing an electrode in electrical communication with the solution;
(c) providing a sacrificial reagent in the solution;
(d) transferring a charge from the sacrificial reagant to the analyte;
(e) transferring a charge from the analyte to the electrode, thereby generating an electrical signal;
(f) repeating steps (d) and (e) to amplify the electrical signal; and
(g) measuring the amplified electrical signal.

2. The method of claim 1 wherein the electrode further comprises a coating layer adjacent to the electrode, the coating layer having an upper surface in contact with the solution and a lower surface in electrical communication with the electrode, the coating layer being capable of conducting a charge from the analyte to the upper surface to the electrode.

3. The method of claim 1 wherein the analyte is a derivative of ferrocene.

4. The method of claim 3 wherein the analyte is hydroxymethylferrocene (HMFc).

5. The method of claim 3 in which the sacrificial reagant is a ferrocyanide.

6. The method of claim 1 wherein the signal is amplified by a factor of at least one thousand.

7. The method of claim 1 in which the solution is provided in a fluid delivery system that comprises a flowing stream.

8. The method of claim 7 in which the fluid delivery system further comprises an analytical separation column and a detector.

9. The method of claim 2 in which the analyte is adsorbed upon the upper surface of the coating layer at least during step (e).

10. The method of claim 2 in which the analyte is adsorbed upon the upper surface of the coating layer during both step (d) and (e).

11. The method of claim 2 in which the transfer of electrical charges from the sacrificial reagent directly to the upper surface of the coating layer is minimized.

12. A method of detecting relatively low levels of an analyte in solution by amplifying amperometric signals, comprising:
(a) providing a solution having an analyte therein, the analyte comprising a ferrocene derivative;
(b) providing an electrode in electrical communication with the solution, the electrode having a coating layer affixed thereon, the coating layer having an upper surface in contact with the solution and a lower surface affixed to the electrode;
(c) providing a sacrificial reagent in the solution;
(d) transferring a charge from the sacrificial reagent to the analyte;
(e) transferring a charge from the analyte to the upper surface of the coating layer;
(f) transferring a charge from the coating layer to the electrode, thereby generating an electrical signal; and
(g) repeating steps (d)-(f) to amplify the electrical signal.

13. The method of claim 12 further comprising the step of:
(h) measuring the amplified electrical signal.

14. The method of claim 12 further wherein the solution is employed in a flowing stream.

15. The method of claim 12 in which the sacrificial reagent comprises a ferrocyanide, further wherein the direct transfer of a charge from the ferrocyanide to the upper surface of the coating layer is minimized or eliminated.

16. The method of claim 12 in which the analyte is affixed to the upper surface of the coating layer during steps (d) and (e).

17. The method of claim 12 in which coating layer is a monolayer.

18. The method of claim 12 in which the ferrocene derivative further comprises a tagged biomolecule, the biomolecule being selected from the group of comprising: proteins, enzymes, oligopeptides, oligonucleotides, and antibodies.

19. The method of claim 18 wherein the ferrocene derivative comprises a ferrocene moiety attached to a strand of deoxyribonucleic acid (DNA).

20. A bioaffinity ligand binding assay method that employs tagged or labeled analytes of ferrocene to detect DNA, comprising:
(a) providing a labeled DNA strand having a ferrocene moiety;
(a) generating a capture DNA strand on a solid bead, the capture DNA strand being combined with the labeled DNA strand;
(c) exposing the capture DNA strand to a target DNA strand;
(d) displacing labeled DNA strand from the capture DNA strand;
(e) attaching the target DNA strand to the capture DNA strand; and
(f) detecting the displaced labeled DNA strand by electrochemical detection methods.

21. A method of determining the number of ferrocene tagged biomolecules in solution at low detection levels, comprising
(a) providing at least one ferrocene group;
(b) linking the ferrocene group with a biomolecule;
(c) tagging the ferrocene group;
(d) injecting the tagged ferrocene group, with attached biomolecule, into a flow stream;
(e) selectively detecting the tagged ferrocene groups; and
(f) determining the amount of biomolecules in solution.
